(19) United States
(12) Patent Application Publication
(10) Pub. No.: US 2011/0236490 A1
(43) Pub. Date: Sep. 29, 2011

(54) TREATMENT OR PREVENTION OF VIRAL INFECTION BY CHLORINATION

(75) Inventor: Emmett M. Cunningham, Phoenix, AZ (US)

(73) Assignee: NEVOA SOLUTIONS, Scottsdale, AZ (US)

(21) Appl. No.: 13/133,882

(22) PCT Filed: Dec. 18, 2009
(86) PCT No.: PCT/US09/68643

§ 371 Date: Jun. 9, 2011

Related U.S. Application Data
(60) Provisional application No. 61/201,990, filed on Dec. 18, 2008.

(51) Int. Cl. A61K 9/14; A61K 33/20; A61P 31/12; A61P 31/16

(52) U.S. Cl. 424/489; 424/661

(57) ABSTRACT

The invention provides methods and systems for treating or preventing viral infection. In one embodiment, the invention provides a method of treating or preventing viral infection in an individual, the method comprising: administering to the individual, via at least one nasal cavity, nebulized ECAW containing a quantity of hypochlorous acid (HOCI) and a quantity of hypochlorite ion (OCI).

### CROSS-REFERENCE TO RELATED APPLICATIONS

This application claims the benefit of co-pending U.S. Provisional patent application No. 61/201,990, filed 18 Dec. 2008, which is hereby incorporated herein.

### TECHNICAL FIELD

The present invention relates generally to the treatment or prevention of infection and, more particularly, to the treatment or prevention of viral infection by chlorination using a liquid or gelled Electro-Chemically Activated Water (ECAW).

### BACKGROUND

Viral infections in humans include the common cold, primarily caused by rhinoviruses, picornaviruses, and coronaviruses, although over 200 different viral types are known to cause colds. Other viral infections include influenza and infectious diseases with similar symptoms. Influenza viruses known to infect humans include three genera: influenza virus A (including the species H1 N1, H2N2, H3N2, H5N1, H7N7, H1N2, N9N2, H7N2, H7N3, and H10N7), influenza B (consisting a single species), and influenza C (consisting of a single species). Collectively, colds, influenza, and infectious diseases with similar symptoms are referred to as influenza-like illness.

Often, viral infection begins with deposition of a virus into the front of the nasal passages. This can occur via contaminated fingers, inhalation of droplets containing the virus, or other mechanisms. Such deposition by as few as one to 30 viral particles is sufficient to produce infection in the individual. Other routes of infection begin, for example, at the eye of an individual.

From the front of the nasal passages, the virus is transported to the back of the nose and onto the adenoid area, often by the mere passage of air during inhalation. The virus then attaches to surface receptors (e.g., Inter-Cellular Adhesion Molecule 1 (ICAM-1) in the case of viruses causing the common cold) on individual cells, enters the cells, replicates, and ruptures the cell, spreading the virus to other cells and repeating the infection cycle. Replication of a virus causing the common cold typically begins eight to 12 hours after infection, with symptoms beginning between 10 hours and five days post-infection and peaking two to three days after onset.

Replication and infection by influenza viruses are similar to those of the common cold. Many influenza viruses attach to a surface receptor in cells of the nose, throat, or lungs via a glycoprotein (hemagglutinin) on the outside of the virus particle. The virus particle then enters the cell, replicates, and is released from the cell. The replication cycle is similar to that of cold viruses, although symptoms typically occur more quickly, last longer, and are more severe.

Influenza is a seasonal disease, periodically resulting in endemic or pandemic outbreaks. In some cases, influenza infection results in life-threatening complications.

While infection by cold virus typically results in much less severe consequences in terms of an individual's health, the overall economic cost of the common cold, including the costs of treatment and lost productivity, is estimated to be over $20 billion per year in the United States alone. In addition, the widespread but futile use of antibiotics to treat cold symptoms has greatly increased antibiotic resistance, the consequences of which, in terms of both dollars and lives, are difficult or impossible to accurately estimate.

Known methods of treating symptoms of cold and influenza, such as the use of saline nasal sprays, dry nasal passages and often lead to irritation. In addition, the use of saline sprays alone is believed to provide a more hospitable environment for viral replication.

### SUMMARY

The invention provides methods and systems for treating or preventing viral infection.

In one embodiment, the invention provides a method of treating or preventing viral infection in an individual, the method comprising: administering to the individual, via at least one nasal cavity, nebulized ECAW containing a quantity of hypochlorous acid (HOCI) and a quantity of hypochlorite ion (OCI⁻).

In another embodiment, the invention provides a system for treating or preventing viral infection in an individual, the system comprising: a nebulizing device; and a quantity of ECAW containing hypochlorous acid (HOCI) and hypochlorite ion (OCI⁻).

In still another embodiment, the invention provides a method of treating or preventing infection in an individual, the method comprising: administering to the individual, via at least one nasal cavity, a quantity of liquid or gelled ECAW containing a quantity of hypochlorous acid (HOCI) and a quantity of hypochlorite ion (OCI⁻).

In still another embodiment, the invention provides a system for treating or preventing viral infection in an individual, the system comprising: a quantity of liquid or gelled ECAW containing hypochlorous acid (HOCI) and hypochlorite ion (OCI⁻); and a delivery device for introducing the liquid or gelled ECAW into a nasal cavity of an individual.

The illustrative aspects of the present invention are designed to solve the problems herein described and other problems not discussed, which are discoverable by a skilled artisan.

### DETAILED DESCRIPTION

The invention provides methods and systems for treating or preventing viral infection by chlorination using a liquid or gelled ECAW. In some embodiments, the liquid or gelled ECAW may be nebulized.

As used herein, the terms “treating,” “treat,” “treatment,” “prevent,” “prevention,” and “preventing” refer to an ameliorative or prophylactic effect on viral count, viral infection, or a symptom of viral infection.

An ECAW suitable for use in practicing various embodiments of the invention may be produced, for example, by electrolysis of a dilute salt solution, such as by passing such a solution through an electrolytic cell. This and other techniques are known and will not be described further herein. The primary active component of such an ECAW is hypochlorous acid (HOCI), a potent antimicrobial effective in rapidly destroying bacteria, viruses, and spores. The hypochlorie ion (OCI⁻) is typically also found in such an ECAW. It, too, is a potent antimicrobial, although its antimicrobial activity is generally less than that of HOCI.

In some embodiments of the invention, the ECAW contains between about 35 ppm and about 200 ppm HOCI and between about 35 ppm and about 200 ppm OCI⁻, depending on the pH of the solution. In some embodiments, the ECAW according to the invention has a pH between about 5 and about 8.

In some embodiments of the invention, the ECAW is used in a liquid or gelled form. That is, the ECAW may be used in a free-flowing form or in a more viscous form. Any known or later-developed technique for increasing the viscosity of a liquid may be employed to produce a gelled ECAW suitable for use in practicing embodiments of the invention.

Viral infection may be treated or prevented, according to some embodiments of the invention, by administering the liquid or gelled ECAW to an individual via a nasal cavity. For example, the liquid or gelled ECAW may be applied to a swab or contained within a syringe, which is then used to introduce the ECAW to the nasal cavity.

Contact with the liquid or gelled ECAW has been shown to kill various viruses within seconds. As such, administration of the liquid or gelled ECAW to the nasal cavity will kill virus particles within the nasal cavity before their infection of cells in the adenoid area. In addition, inhalation will carry at least a portion of the liquid or gelled ECAW deeper into the nasal cavity and to the adenoid area, where the antiviral activity of the HOCI and OCI⁻ will kill virus particles that may have already reached the adenoid area.

In other embodiments, the liquid or gelled ECAW may be administered in a nebulized form. As used herein, “nebulized” refers to micronized, atomized, vaporized, or aerosolized particles or a fine spray of a liquid or gel. In some embodiments, a nebulized form of the liquid or gelled ECAW comprises micro particles having a diameter between about 5 microns and about 1000 microns.

Various techniques and devices for nebulizing liquids and gels are known and may be employed in practicing embodiments of the invention. Suitable nebulizing devices include, for example, piezoelectric nebulizers, spray bottles, metered spray pumps, metered-dose inhalers, and bag-on-valve spray cans. Other devices and various techniques for nebulizing a liquid or gel will be known to one skilled in the art and are within the scope of the present invention.

Administration of the ECAW in nebulized form facilitates more extensive delivery of the ECAW than is typically achieved through administration of a liquid or gelled ECAW in unnebulized form. For example, inhalation during administration of the nebulized ECAW will carry the ECAW into the nasal passages to the adenoid area and into the lungs.

While described herein as useful in treating or preventing viral infection, embodiments of the present invention may also be employed in killing or destroying bacterial cells and spores. In addition, while described herein as used to treat or prevent viral infection in an individual by administering an ECAW to such an individual, embodiments of the invention are equally applicable to the disinfection of surfaces and atmospheres. For example, a liquid or gelled ECAW such as those described above may be applied to a cloth or wipe for disinfecting a surface or released as a fog or mist to disinfect an atmosphere.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A method of treating or preventing viral infection in an individual, the method comprising:
administering to the individual, via at least one nasal cavity, nebulized Electro-Chemically Activated Water (ECAW) containing a quantity of hypochlorous acid (HOCI) and a quantity of hypochlorite ion (OCI⁻).

2. The method of claim 1, further comprising:
nebulizing a quantity of at least one of the following: liquid ECAW containing the HOCI and the OCI⁻ or gelled ECAW containing the HOCI and the OCI⁻.

3. The method of claim 2, wherein nebulizing includes forming micro particles of the liquid ECAW using at least one device selected from a group consisting of: a piezoelectric nebulizer, a spray bottle, a metered spray pump, a metered-dose inhaler, and a bag-on-valve spray can.

4. The method of claim 1, wherein the viral infection includes infection by at least one virus selected from a group consisting of: a virus causing the common cold and an influenza virus.

5. The method of claim 4, wherein the virus causing the common cold is selected from a group consisting of: rhinoviruses, picornaviruses, and coronaviruses.

6. The method of claim 4, wherein the influenza virus is selected from a group consisting of: influenza virus A, influenza virus B, and influenza virus C.

7. The method of claim 4, wherein at least one of the quantity of HOCI and the quantity of OCI⁻ is capable of preventing replication of the virus causing the common cold, the influenza virus, or both.

8. The method of claim 1, wherein the nebulized ECAW has a pH between about 5 and about 8.

9. A system for treating or preventing viral infection in an individual, the system comprising:
a nebulizing device; and
a quantity of Electro-Chemically Activated Water (ECAW) containing hypochlorous acid (HOCI) and hypochlorite ion (OCI⁻).

10. The system of claim 9, wherein the quantity of ECAW includes liquid ECAW.

11. The system of claim 9, wherein the quantity of ECAW includes gelled ECAW.

12. The system of claim 9, wherein the nebulizing device is capable of nebulizing the ECAW to form micro particles of ECAW.

13. The system of claim 12, wherein the nebulizing device is selected from a group consisting of: a piezoelectric nebulizer, a spray bottle, a metered spray pump, a metered-dose inhaler, and a bag-on-valve spray can.

14. The system of claim 12, wherein the micro particles have a diameter between about 5 microns and about 1000 microns.

15. The system of claim 9, wherein the viral infection includes infection by at least one virus selected from a group consisting of: a virus causing the common cold and an influenza virus.

16. The system of claim 15, wherein the virus causing the common cold is selected from a group consisting of: rhinoviruses, picornaviruses, and coronaviruses.

17. The system of claim 15, wherein the influenza virus is selected from a group consisting of: influenza virus A, influenza virus B, and influenza virus C.

18. The system of claim 15, wherein at least one of the HOCI and the OCI⁻ is capable of preventing replication of the virus causing the common cold, the influenza virus, or both.

19. The system of claim 9, wherein the quantity of ECAW has a pH between about 5 and about 8.

20. A method of treating or preventing infection in an individual, the method comprising:
administering to the individual, via at least one nasal cavity, a quantity of liquid or gelled ECAW containing a quantity of hypochlorous acid (HOCI) and a quantity of hypochlorite ion (OCI⁻).

21. The method of claim 20, wherein administering includes introducing into the at least one nasal cavity the quantity of liquid or gelled ECAW using at least one of a syringe and a swab.

22. The method of claim 20, wherein the liquid or gelled ECAW has a pH between about 5 and about 8.

23. A system for treating or preventing viral infection in an individual, the system comprising:
a quantity of liquid or gelled ECAW containing hypochlorous acid (HOCI) and hypochlorite ion (OCI); and
a delivery device for introducing the liquid or gelled ECAW into a nasal cavity of an individual.

24. The system of claim 23, wherein the delivery device is selected from a group consisting of: a syringe and a swab.
